(19) United States
(12) Patent Application Publication
(10) Pub. No.: US 2005/0080479 A1
(43) Pub. Date: Apr. 14, 2005

(54) Expandable endovascular stent

(76) Inventors: James Q. Feng, Fairport, NY (US); Hui Sun, Fairport, NY (US)

Correspondence Address: James Q. Feng, 18 County Clare Crescent, Fairport, NY, 14450, US

(21) Appl. No.: 10/671,691

(22) Filed: Sep. 29, 2003

(51) Int. Cl. A61F 2/06

(52) U.S. Cl. 623/1.15

(57) ABSTRACT

Disclosed herein is a tubular endovascular stent comprising a plurality of annular segments connected by one or more bridging elements. Each annular segment takes forms of periodic wavelets with a plurality of alternating symmetric peaks and valleys, preferably consisting of circular arc segments of large radii connected tangentially with straight segments to minimize stress concentration when the stent undergoes radial deformation, transverse to the longitudinal axis of the stent. The points of connection between the bridging elements and adjacent annular segments are so designed that deformations of the bridging elements remain negligible as the stent deforms radially, namely, the longitudinal dimension of the stent does not vary during the radial expansion or contraction of the stent. Hence, the radial strength and the longitudinal flexibility of the stent made according to the principles disclosed by the present invention can be independently controlled by the design parameters for the annular segments and bridging elements, without compromising the longitudinal dimensional stability of the stent. Since stress concentration and deformation in the stent can lead to restenosis, stent made from the invention disclosed here can reduce the probability of restenosis.

### REFERENCES CITED U.S. PATENT DOCUMENTS

U.S. PATENT DOCUMENTS

| | | |
|---|---|---|
| 3,868,956 | March 1975 | Alfidi et al. |
| 4,503,569 | March 1985 | Dotter |
| 4,768,507 | September 1988 | Fischell |
| 4,776,337 | October 1988 | Palmaz |
| 4,800,882 | January 1989 | Gianturco |
| 4,830,003 | May 1989 | Wolff et al. |
| 4,856,516 | August 1989 | Hillstead |
| 4,886,062 | December 1989 | Wiktor |
| 5,383,892 | June 1995 | Cardon et al |
| 5,449,373 | September 1995 | Pinchasik et al |
| 5,695,516 | December 1997 | Fischell et al |
| 5,725,548 | March 1998 | Jayaraman |
| 5,733,303 | March 1998 | Israel et al |
| 5,733,330 | March 1998 | Cox |
| 5,776,181 | July 1998 | Lee et al |
| 5,913,895 | June 1999 | Burpee et al |
| 5,938,697 | August 1999 | Killion |

### FIELD OF THE INVENTION

The present invention relates to a radially expandable endoprosthesis device, and in particular to an expandable endovascular stent for implantation within a body vessel such as coronary or peripheral arteries or bile duct or urinary tract to widen a stenosis or open a blockage in the body vessel.

### BACKGROUND OF THE INVENTION

Endovascular stenting is a method for inserting a prosthesis into a body vessel to widen a stenosis or to open a collapsed vessel wall, therefore to prevent restenosis or the wall from recollapsing into the vessel. The expandable endovascular stent is typically implanted intraluminally in its compressed or collapsed state using a catheter which is inserted at an easily accessible location and then advanced to the deployment site where the stent is to be radially expanded in situ from its compressed state. The stent may be self-expanded or expanded by inflation of a balloon on which the stent is mounted and carried on the catheter at the deployment site. After deployed in its expanded state, the stent is left in place while the balloon is deflated and withdrawn from the vessel with the catheter. This method is particularly useful for treatment of blocked or narrowed arteries due to stenosis to prevent restenosis following angioplasty in the vascular system. For general information with regard to vascular stents, reference may be made to the “Textbook of Interventional Cardiology” by Eric J. Topol, W. B. Saunders Company, 1999 and, in particular, to Chapters 29-31 of Vol. II about stents.

The prior art dedicated to the present field of invention includes a large number of patent documents as shown, for example, by U.S. Pat. No. 3,868,956 (Alfidi et al.); U.S. Pat. No. 4,503,569 (Dotter); U.S. Pat. No. 4,768,507 (Fischell); U.S. Pat. No. 4,776,337 (Palmaz); U.S. Pat. No. 4,800,882 (Gianturco); U.S. Pat. No. 4,830,003 (Wolff et al.); U.S. Pat. No. 4,856,516 (Hillstead); U.S. Pat. No. 4,886,062 (Wiktor); U.S. Pat. No. 5,383,892 (Cardon et al); U.S. Pat. No. 5,449,373 (Pinchasik et al); U.S. Pat. No. 5,695,516 (Fischell etal); U.S. Pat. No. 5,725,548 (Jayaraman); U.S. Pat. No. 5,733,303 (Israel et al); U.S. Pat. No. 5,733,330 (Cox); U.S. Pat. No. 5,776,181 (Lee et al); U.S. Pat. No. 5,913,895 (Burpee et al); U.S. Pat. No. 5,938,697 (Killion). As described in the prior art, stents have been made in many configurations such as wire-woven mesh, coiled spring, various shaped closed or open cells, combinations of rigid segments and flexible connectors, etc. Many materials including ordinary metals such as stainless steel, shape memory metals such as nitinol, biodegradable plastics, etc. can be used to make stents, as long as they satisfy the requirements of excellent corrosion resistance and biocompatibility. Balloon-expandable stents in general are made from materials of low yield stress and high elastic modulus so that they can be plastically deformed by the inflation of a balloon at manageable balloon pressures. The deployed stent should remain in its expanded shape after the balloon is deflated, except for some slight recoil caused by the elastic portion of the deformation which can be reduced by using materials with high elastic modulus. Self-expandable stents, on the other hand, are typically made from materials of high yield stress and low elastic modulus so that they can be compressed and constrained in a delivery system and then spring back to their preset diameters upon release from the delivery system.

To hold an otherwise blocked or constricted lumen open, a stent is required to possess sufficient radial or hoop strength in its expanded state. It is conceivable that large contact area between the stent surface and the vessel wall can usually help distribute the load effectively, such that the stent radial and hoop strength easily become adequate. However body vessels have a natural tendency of rejecting contact of foreign materials; thus a smaller contact area between the stent surface and vessel wall would be preferred. Furthermore, to facilitate its advancement on the catheter through the lumen, the stent is also desired to be as small and compact as possible in its compressed state. For the convenience of maneuvering the stent through the vasculature, which may consist of significant local curvatures, and for better conforming to any local shape of the vasculature at the deployment site, longitudinal flexibility of the stent is of great importance. To eliminate or minimize abrasion trauma as may be inflicted on the vessel wall during radial expansion of the stent, the longitudinal stability of the stent during its radial expansion, namely the longitudinal dimension of the stent does not tend to change as the stent expands in the radial direction, ought to become a significant factor in the stent configuration design consideration.

A major problem with the stent configurations disclosed by the prior art is that the amount of material needed to ensure its sufficient radial strength severely limits the compactness of the stent in its compressed state for delivery. The difficulty in reducing the amount of material needed to ensure sufficient stent radial strength often comes from non-optimized stent configurations that lead to substantial stress concentration in some small zones as the stent deforms in the radial direction. Localized stress concentration is also known to have a detrimental tendency to lead to stess-induced restenosis in the stented vessel. Furthermore the interdependence and tradeoff between the radial and hoop strength and longitudinal flexibility, which also tend to cause considerable longitudinal contraction or shortening during radial expansion of the stent often leads to undesirable, if not serious, abrasion trauma on the vessel wall.

What has been needed and heretofore unavailable is a stent that has a configuration enabling nearly independent control of longitudinal flexibility and radial strength, where sufficient radial strength is ensured by optimizing the stent configuration for stress distribution and therefore minimizing stress concentration in the stent material the amount of which can thus be reduced to a minimal level, so that it can be easily delivered through tortuous lumen passages while having sufficient radial strength to hold open the body lumen into which it is expanded, and that maintains longitudinal dimension stability during its radial expansion, so that abrasion trauma on the vessel wall is minimized. The present invention satisfies this need.

### SUMMARY OF THE INVENTION

The objective of the present invention is to provide an expandable endovascular stent configuration which minimizes the amount of material required for achieving adequate radial strength of the stent.

The further objective of the present invention is to provide an expandable endovascular stent which has substantial longitudinal flexibility and longitudinal dimensional stability during its radial expansion or contraction to facilitate delivery through tortuous body lumens and to minimize abrasion trauma on the vessel wall while retaining sufficient radial and hoop strength for supporting the vessel wall when implanted therein.

The objectives of the present invention is achieved by designing an expandable endovascular stent comprising a single walled tubular body of a biocompatible material having a plurality of annular segments, which are transverse to the longitudinal axis and have periodic wavelets with a plurality of alternating symmetric peaks and valleys consisting of an arc segment and a straight segment, with the said arc segment having a constant or nearly constant curvature and having the arc angle greater than 180 degrees when the stent is in compressed state, and with the said straight segment being tangentially connected to the said arc segment. The large radii of circular arc segments make it possible to distribute the local curvature variation over large amount of material during radial deformation of the stent, and therefore to eliminate the undesirable stress concentration, because the radial expansion and contraction of the stent correspond to changes in local curvature and the longitudinal projection of the peak-to-valley distance of those periodic wavelets in the annular segments.

In a preferred embodiment of the invention the expandable endovascular stent further comprises bridging elements connected to the annular segments with the connection points located at or near the stress-neutral points, where the said stress-neutral point being located midway between the symmetric peaks and valleys of the wavelets of the annular segments because the longitudinal projections of the middle points between the symmetric peaks and valleys of the wavelets of the annular segments do not change during radial deformation of the stent Another advantage of connecting the bridging elements with annular segments at those stress-neutral points is to reduce the susceptibility of restenosis development, as restenosis is known to occur more likely at the stress concentrated connection points.

According to the design principles disclosed by the present invention, the radial strength and the longitudinal flexibility of the stent can be determined independently by the design parameters for the annular segments and bridging elements, respectively, without compromising longitudinal dimension invariability of the stent.

### BRIEF DESCRIPTION OF THE DRAWINGS

The invention is herein described, by way of example only, with reference to the accompanying drawings, wherein:

FIG. I is a flat layout view of a stent according to the present invention;

FIG. 2 is a flat layout view of the stent shown in FIG. 1 in its compressed (undeployed) state and expanded (deployed) state;

FIG. 3 is a partial section of an annular segment of the stent shown in FIG. 1;

FIG. 4 is a flat layout view of a typical prior art stent in its compressed (undeployed) state and expanded (deployed) state.

### DESCRIPTION OF THE PREFERRED EMBODIMENT

For the purposes of promoting an understanding of the principles of the present invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated herein being contemplated as would normally occur to one skilled in the art to which the invention relates.

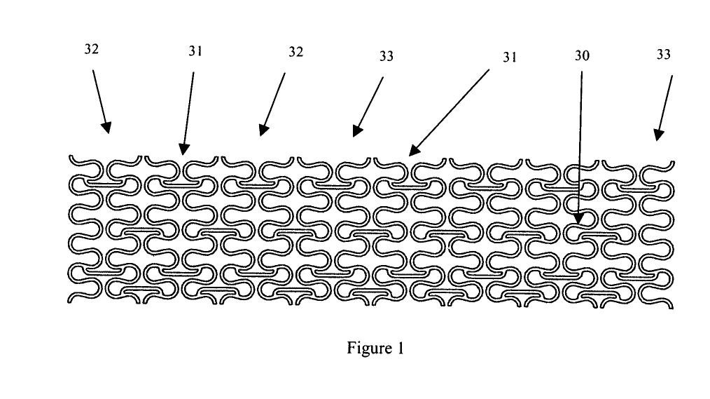

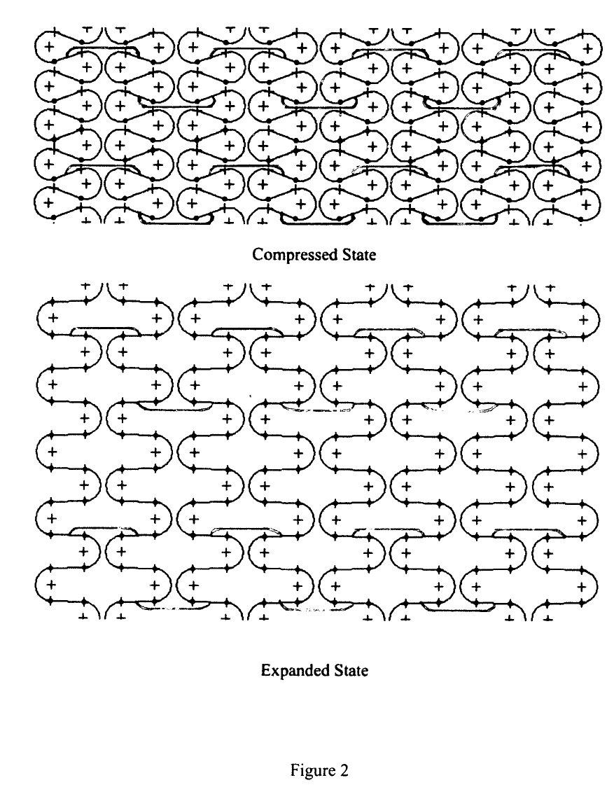

Illustrated in FIGS. 1-2 is a flat layout view of a tubular stent incorporating features of the present invention in its compressed and expanded states, respectively. The tubular stent generally comprises a plurality of annular segments 30, as referred to as “struts” hereafter, and a plurality of connecting elements 31, as referred to as “bridges” hereafter. Said struts are assembled in parallel along the length of the stent.

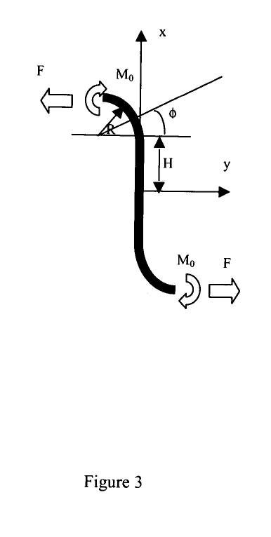

The struts are preferably configured as a plurality of periodic wavelets with symmetric peaks and valleys that are divided into circular-arc segments 32 with a radius R and straight-line segments 33 with a half-length H as detailed in FIG. 3. Said circular-arc segments and said straight segments are preferably connected smoothly (tangent to each other). Based on FIG. 3, a mechanical analysis can be performed as follows.

A radial pressure p in a tubular body vessel of diameter D will result in a net segmental force F in the strut in the hoop (circumferential) direction of the stent, according to the Laplace equation:
F=L p D/2 (1)
where L is the length of bridges, which may become approximately the height of the struts L=2(H+R) (elements 30 or 31 shown in FIG. 1). The segmental hoop stress in the strut becomes maximum at the peaks and valleys (Φ=π/2, cf. FIG. 3) as given by

$${σ}_{max} = \frac{F}{{w}_{s} t} ( 1 + \frac{6 R ( 1 + k )}{{w}_{s}} ) \qquad ( 2 )$$

where Wₛ is the segment width and t the segment thickness (in the radial direction of the stent, with k denoting the ratio H/R (cf. FIG. 3). The first term on the right hand side of equation (2) is a direct result of the segmental force F whereas the usually dominant second term is due to the local bending moment. Using the beam theory with linear elasticity approximation, the local deflection at Φ=π/2 in the hoop direction of the stent due to the segmental hoop stress can be found as

$$d = \frac{π F {R}^{3}}{4 E I} {f}_{0} ( k ) \qquad ( 3 )$$

where El is the bending stiffness with E denoting the Young's modulus and I=w³ₛt/12, and ƒ₀(k) is the geometric factor given by

$${f}_{0} ( k ) = 1 + \frac{8 k}{π} + 2 {k}^{2} + \frac{4 {k}^{3}}{3 π} . \qquad ( 4 )$$

Thus, a strut with m wavelets will have the expansion ratio Q given by

$$Q = \frac{4 m d}{D π} = \frac{m F {R}^{3}}{D E I} {f}_{0} ( k ) \qquad ( 5 )$$

where 4 md is the total displacement of the strut in the circumferential direction of the stent. The stent stress index X defined as the ratio of the maximum stress and the expansion ratio,

$$χ = \frac{{σ}_{max}}{Q} = \frac{D E I}{{w}_{s} t m {R}^{3} {f}_{0} ( k )} ( 1 + \frac{6 R ( 1 + k )}{{w}_{s}} ) \qquad ( 6 )$$

indicates that increasing the radius of the circular-arc segments of the struts, R, can reduce the maximum stress for a given stent expansion ratio. Therefore, with all the design constraints considered, the radius R of said circular-arc segments is preferably made as large as possible to minimize stress concentration as the stent deforms during expansion or contraction or in response to vessel wall pressure variations due to hemodynamics, such as during cycles of diastole and systole. The length 2H of said straight segments can be adjusted according to the required expansion ratio of said stent in the radial direction.

Each bridge consists of mostly a straight segment with curved ends to perpendicularly join with adjacent struts. The locations of joins between bridges and struts are chosen at the stress-neutral points of struts, namely at midway between the symmetric peaks and valleys of the wavelets of struts where the local displacement in the axial direction of the stent (x-direction in FIG. 3) diminishes theoretically as the stent deforms radially. Therefore, said bridges are substantially stress free during radial deformation of said stent, and the length L of each bridge does not vary with radial expansion and contraction of the stent. This feature minimizes the longitudinal dimension variation of said stent, namely said stent can maintain the same length whether in compressed or expanded states or during its radial deformation, in contrast to stent configurations in the prior art.

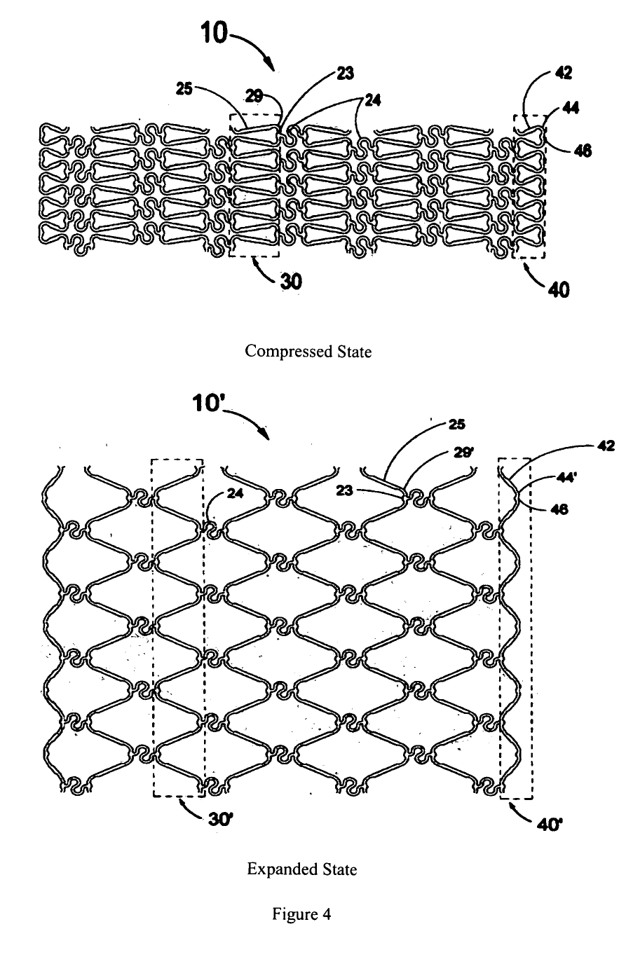

An example of stent designs in the prior art is shown in FIG. 4, from which it is easy to understand that stents of this prior art design will undergo significant shortening when the stent is expanded; in particular, section 30, going through both radial expansion and longitudinal shortening, is deformed into section 30′ and the bridging elements connecting adjacent sections at their peaks and valleys accumulate all the local shortening of each section in the longitudinal direction into an overall longitudinal shortening of the stent.

Moreover, the length L of each bridge and the number of bridge elements can also be adjusted to achieve desired longitudinal flexibility for intraluminal delivery without compromising the radial strength of said stent.

Although FIGS. 1-2 show an open cell design with only two bridges connecting the adjacent struts with several straight segments in each strut not connected by any bridges, more bridges can be used to connect adjacent struts if so desired. Even a close cell stent can be envisioned that all the stress-neutral points at the midway of the symmetric peaks and valleys of the wavelets of the struts are connected with bridges.

The tubular design of the stent may allow the plastic deformation of the material when the stent is expanded by a balloon catheter. Such plastic deformation can maintain the stent in its expanded position and resist collapsing in the vessel wherein the stent is implanted. With super-elastic NiTi alloys, e.g., Nitinol, the expansion occurs when the compressing stress is removed so as to allow the phase transformation from austenite back to martensite with the stent expansion.

The tubular stent incorporating features of the present invention can be produced by laser cutting from a hollow cylinder. Typically using Nd:YAG lasers, the laser cutting method can confine kerf widths to less than 20 micrometers. Thus, intricate patterns can be created on a wide range of hollow cylinders with diameter greater than 0.5 mm. Balloon-expandable stents can be cut in their compressed configuration, and then be deburred and electropolished. Self-expanding nitinol stents can be cut either in their compressed configuration, which requires post-cutting expansion and shape-setting, or in their expanded configuration, and finally be deburred and electropolished.

Also, the stent may be coated with an anticoagulating medication substance, such as heparin, or other bio-absorbable materials. Consequently, blood clotting can be prevented/reduced when the stent is implanted in a blood vessel.

The struts of the stent are preferred to consist of circular arc segments of large radii connected smoothly with straight segments, however, variation of this design does exist and for example, the circular arc segments can be modified to as arc segments of varied curvatures.

The stent may be dimensioned to fit within the intended vessel for insertion and engage the vessel wall when expanded. A typical stent for an artery may have a diameter of between 1.5 millimeter and 3.5 millimeter during insertion and may have a diameter of between 2 millimeter and 12 millimeter when expanded.

Various other modifications, adaptations, and alternative designs are possible in light of the above teachings. Therefore, it should be understood at this time that within the scope of the appended claims the invention may be practiced otherwise than as specifically described herein.

1. An expandable endovascular stent for implanting in a body vessel comprising a single walled tubular body of a biocompatible material having a plurality of annular segments, said annular segments being transverse to the longitudinal axis and having periodic wavelets with a plurality of alternating symmetric peaks and valleys consisting of an arc segment and a straight segment, the said arc segment having a constant or nearly constant curvature, the straight segment being tangentially connected to the arc segment.

2. An expandable endovascular stent according to claim 1 further comprises bridge elements.

3. An expandable endovascular stent according to claim 2 wherein the said bridging elements are connected to the annular segments with the connection points located at or near the stress-neutral points, the said stress-neutral point being located midway or close to midway between the symmetric peaks and valleys of the wavelets of the annular segments.

4. An expandable endovascular stent according to claim 1 wherein the stent longitudinal dimension is substantially the same in the expanded state, the compressed state and any other states between.

5. An expandable endovascular stent according to claim 1 wherein the said annular segment is designed according to the folowing formula I for stress index X:
$$χ = \frac{{σ}_{max}}{Q} = \frac{D E I}{{w}_{s} t m {R}^{3} {f}_{0} ( k )} ( 1 + \frac{6 R ( 1 + k )}{{w}_{s}} ) \qquad ( I )$$
which is defined as the ratio of the maximum segmental stress (in circumferential direction) in the strut at the peaks and valleys (Φ=π/2, cf. FIG. 3) τₘₐₓ and expansion ratio Q, where
$${σ}_{max} = \frac{F}{{w}_{s} t} ( 1 + \frac{6 R ( 1 + k )}{{w}_{s}} ) , and F = L p D / 2$$
denoting the net segmental force in the strut in the hoop (circumferential) direction of the stent as the result of a radial pressure p in a tubular body vessel of diameter D according to the Laplace equation, where L is the length of bridges, which may become approximately the height of the struts L=2(H+R) (elements 30 or 31 shown in FIG. 1), wₛ the segment width, t the segment thickness, k=H/R the ratio of the half length of straight segment H and the radius of the arc curvature R, and the expansion ratio Q is given by
$$Q = \frac{4 m d}{D π} = \frac{m F {R}^{3}}{D E I} {f}_{0} ( k )$$
with d denoting the deflection of the half wavelets of annual segment
$$d = \frac{m F {R}^{3}}{4 E I} {f}_{0} ( k ) ,$$
m being the number of the wavelets, El the bending stiffness of the annual segment, and ƒ₀(k) the geometric factor given by
$${f}_{0} ( k ) = 1 + \frac{8 k}{π} + 2 {k}^{2} + \frac{4 {k}^{3}}{3 π} .$$

6. An expandable endovascular stent according to claim 1 wherein said stent is expandable by a balloon catheter.

7. An expandable endovascular stent according to claim 1 where in said stent is made of a bio-compatible material capable of elastic and plastic deformation.

8. An expandable endovascular stent according to claim 7 wherein said bio-compatible material is stainless steel.

9. An expandable endovascular stent according to claim 7 wherein said bio-compatible material is gold.

10. An expandable endovascular stent according to claim 7 wherein said bio-compatible material is a nickel titanium alloy

11. An expandable endovascular stent according to claim 10 wherein said nickel titanium alloy is nitinol.

12. An expandable endovascular stent according to claim 1 wherein said stent is coated with a substance that prevents blood coagulation.

13. An expandable endovascular stent according to claim 2 wherein said annular segments are connected by bridge elements to form stents having close cells.

14. An expandable endovascular stent according to claim 2 wherein said annular segments are connected by bridge elements to form stents having open cells.

15. An expandable endovascular stent according to claim 1 wherein said arc segment has an arc angle more than 180 degrees when the stent is in the compressed state.

16. An expandable endovascular stent according to claim 1 wherein the curvatures of the arc segments are varied.
